(19) United States
(12) Patent Application Publication
(10) Pub. No.: US 2024/0389874 A1
(43) Pub. Date: Nov. 28, 2024

(54) VITAL SENSOR AND METHOD FOR OPERATING A VITAL SENSOR

(71) Applicant: ams-OSRAM International GmbH, Regensburg (DE)

(72) Inventors: Thomas SCHWARZ, Regensburg (DE); Michael JOBST, Wiesenfelden (DE); Sergey KUDAEV, Regensburg (DE)

(21) Appl. No.: 18/695,465

(22) PCT Filed: Sep. 26, 2022
(86) PCT No.: PCT/EP2022/076726

§ 371 Date: Mar. 26, 2024

(30) Foreign Application Priority Data
Sep. 27, 2021 (DE) 10 2021 124 942.2

(51) Int. Cl. A61B 5/024; A61B 5/00

(52) U.S. Cl. CPC A61B 5/02427; A61B 5/7264; A61B 2560/0462; A61B 2562/0233

(57) ABSTRACT

A vital sensor includes at least one pixelated emitter array with first and second pixels. The pixels are configured to emit light of a wavelength range in the direction of a projection surface. The vital sensor also includes an optical element, arranged between the at least one pixelated emitter array and the projection surface, and which is configured to direct light of the first pixel onto a first region of the projection surface and light of the second pixel onto a second region of the projection surface which differs from the first region. The vital sensor further includes a photodetector configured to detect the light emitted by the pixels and reflected on the projection surface. The vital sensor additionally includes an evaluation unit configured to control the first pixel and the at least one second pixel in a pulsed and time-sequential manner to determine a first reference value.

The present application claims the priority of the German patent application No. 10 2021 124 942.2 of Sep. 27, 2021, the disclosure of which is hereby incorporated into the present application.

A vital sensor, in particular a pulse sensor, is specified. In addition, a method for operating a vital sensor, in particular a pulse sensor, is provided.

Publication WO 2018/206391 A1 relates to a sensor module for pulse oximetry.

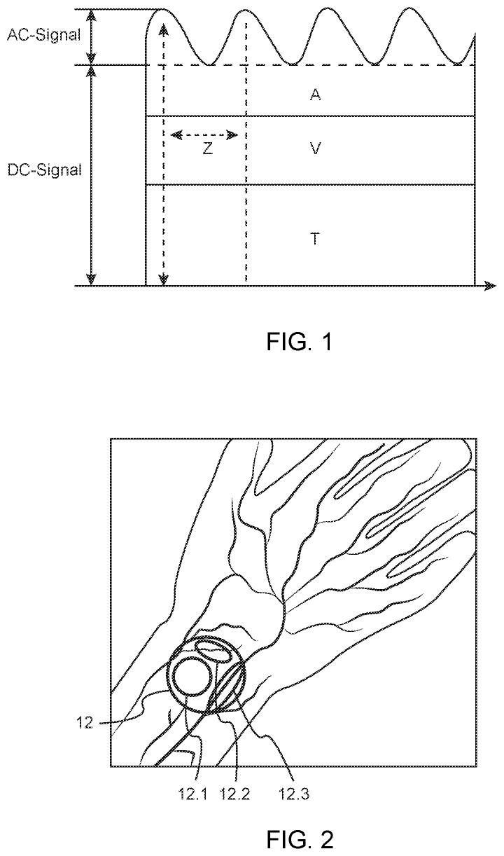

The measurement of vital functions (VSM: vital sign monitoring) such as heart rate, heart rate variability or oxygen content in the blood can be carried out using a PPG (photoplethysmogram), for example. A photoplethysmogram is an optically obtained plethysmogram that can be used to record blood volume changes in the microvascular tissue bed. A PPG is often obtained with a pulse oximeter that illuminates the skin and measures changes in light absorption based on the light reflected back from the skin. As shown as an example in FIG. 1, the reflected-back signal detected by a photodiode over the time t consists of light reflections from the tissue or skin T, light reflections from the blood in the veins V, which is particularly poor in oxygen, and light reflections from the blood in the arteries A, which is particularly rich in oxygen. However, only the component of the light reflected back by the arterial blood or the change in the reflection of the arterial blood within a pulse measurement cycle Z is of interest for measuring the heart rate, for example, in particular the pulsating component (AC signal) of the measured signal. However, the measured pulsating t (AC signal) compared to the measured DC component (DC signal) of the detected signal is usually only a few % (<10%). However, a good signal amplitude of the AC component is important for reliable measurement and, in particular, high measurement accuracy of vital functions.

In addition, the use of a vital sensor, for example in wearable devices such as smartphones, chest straps for fitness trackers, smartwatches or fitness wristbands, requires low energy consumption in order to increase the battery life of the devices.

One task to be solved is to specify a vital sensor, in particular a pulse sensor, which works reliably, has a high measuring accuracy and whose energy consumption is low.

This task is solved, among other things, by a vital sensor, in particular a pulse sensor, with the features of the independent patent claim. Preferred embodiments are the subject of the remaining claims.

According to at least one first embodiment, a vital sensor, in particular a pulse sensor, comprises at least one pixelated emitter array with a first and at least one second pixel, each of which is configured to emit light of a wavelength range in the direction of a projection surface. The projection surface can in particular be the skin, for example on the wrist, a fingertip or an ear of a human wearer of the vital sensor.

Furthermore, the vital sensor comprises at least one optical element which is arranged between the at least one pixelated emitter array and the projection surface and which is configured to direct light from the first pixel onto a first region of the projection surface and light from the at least one second pixel onto a second region of the projection surface which differs from the first. The at least one optical element is configured in particular to project or direct the light emitted by the at least one pixelated emitter array or the light emitted by the pixels of the pixelated emitter array onto different areas of the projection surface, i.e. pixelated, onto the projection surface.

In addition, the vital sensor comprises at least one photodetector, which is configured to detect the light emitted by the pixels and reflected on the projection surface, and an evaluation unit, which is configured to control the first and the at least one second pixel in a pulsed and time-sequential manner to determine a first reference value, in particular separately for each pixel.

The core idea of at least one embodiment is to illuminate essentially only those areas of the projection surface or the skin of a human wearer of the vital sensor for measuring a vital parameter of the wearer that have a high arterial density or arteries close to the skin surface in order to obtain a good AC/DC signal ratio. Areas of the skin with a medium or low arterial density are preferably not illuminated for measuring the vital function. This makes it possible on the one hand to improve the AC/DC signal ratio of the measured signal and on the other hand to save energy.

If you look at the distribution of the arteries, e.g. on the wrist of a human wearer of the vital sensor (see FIG. 2, for example), you will see that the arteries in the hand and other parts of the body are both very wide and fine and, above all, very branched. However, the density of the arteries in the hand varies depending on the area. Within an area on the wrist where, for example, a wristwatch is in contact with the skin, there are areas with low, medium and high arterial density. If the areas with a high arterial density are identified by means of a reference measurement, in particular on the basis of the first reference value, it is possible to illuminate only these areas to measure a vital function in order to improve the AC/DC signal ratio of the measured signal on the one hand and to save energy on the other.

According to at least one embodiment, the evaluation unit can be configured accordingly to control at least one of the first and the at least one second pixel for measuring a vital parameter, in particular the pulse rate, of the human wearer on the basis of the first reference value.

The first reference value can, for example, have a value for the AC-DC ratio measured by the at least one photodetector for each pixel that was measured after irradiating an area of the projection surface for this pixel. If the first reference value is greater than or equal to a predefined threshold value, i.e. has a “good” AC-DC ratio, this pixel is used for a subsequent measurement of a vital parameter of the human wearer. However, if the first reference value is smaller than the predefined threshold value, i.e. has a “bad” AC-DC ratio, the corresponding pixel is not used for the measurement of the vital parameter. This means that only the pixels that provide a “good” AC-DC ratio for measuring the vital parameter can be used or energized for measuring the vital parameter. The first reference value can, for example, be the current intensity I measured in the at least one photodetector per cross-sectional area A through which current flows, i.e. the current density, for each pixel. A “good” AC-DC ratio can be characterized by the fact that the current density for a pixel is between 100 and 500 A/cm². Additionally or alternatively, an algorithm that adapts to the measurement can also be used for data evaluation. This can include a “machine learning” process, whereby the sensor automatically adapts to its carrier and thus pre-processes the measurement data in the first derivation.

According to at least one embodiment, the evaluation unit can accordingly be configured to sequentially control all pixels in a first step in order to determine a first reference value for each pixel of the at least one pixelated emitter array on the basis of a signal sequentially detected by the at least one photodetector. Furthermore, the evaluation unit can be configured to use the determined first reference values in a second step to control the pixels for measuring a vital parameter of the human wearer of the vital sensor, in particular simultaneously, for which the first reference value exceeds a predefined threshold value.

According to at least one embodiment, the vital sensor is configured to determine a second reference value during the measurement of the vital parameter, and the evaluation unit is configured to control the first and at least one second pixel in a pulsed and time-sequential manner on the basis of the second reference value. The second reference value can, for example, have a value for the AC-DC ratio measured by the at least one photodetector, which is measured during the irradiation of the projection surface for the pixels active during the measurement of the vital parameter. If the second reference value changes during the measurement of the vital parameter, for example due to slipping or tilting of the vital sensor on the skin, or if the second reference value in particular has a lower value than a predefined threshold value, i.e. has a “poor” AC-DC ratio, the vital sensor is configured to perform a new reference measurement for each pixel of the at least one pixelated emitter array to determine the first reference value. On the basis of these re-measured first reference values, the evaluation unit is in turn configured to simultaneously control the pixels that are used to measure the vital parameter in order to perform a new measurement of the vital parameter.

According to at least one embodiment, the at least one optical element comprises at least one of a refractive lens, in particular a spherical lens; a Fresnel step lens; a diffractive optical element, in particular a diffractive lens; and a lens made of a metamaterial. The at least one optical element can be configured in particular to deflect the light emitted by the individual pixels of the pixelated emitter array and to illuminate areas on the skin that are separated from each other or at most slightly overlap. The at least one optical element should be as small as possible.

According to at least one embodiment, an aperture is arranged in front of each pixel of the pixelated emitter array, which is configured to limit the cross-section of the light emitted by the pixels, in particular to limit it to a point of light. This makes it possible to focus the light emitted by the individual pixels of the pixelated emitter array onto an area of the at least one optical element, so that the light emitted by the individual pixels of the pixelated emitter array can be deflected separately by means of the optical element.

According to at least one embodiment, the at least one optical element comprises at least one spherical lens. The at least one spherical lens can, for example, be configured to deflect the light of at least one pixel of the at least one pixelated emitter array onto a region of the projection surface.

According to at least one embodiment, the at least one optical element comprises a plurality of spherical lenses. Alternatively or additionally, however, the at least one optical element may also comprise at least one thick lens and/or at least one Fresnel step lens and/or at least one diffractive lens.

According to at least one embodiment, at least two pixels of the pixelated emitter array are associated with the at least one spherical lens, wherein the at least one spherical lens is configured to deflect or project the light of the at least two pixels onto different areas of the projection surface. However, it is also conceivable that several pixels of the pixelated emitter array are assigned to the at least one spherical lens, whereby the at least one spherical lens is configured to deflect or project the light of the several pixels onto different areas of the projection surface.

According to at least one embodiment, the at least one optical element comprises a number of spherical lenses which corresponds to the number of pixels of the pixelated emitter array, wherein a spherical lens is assigned to each pixel of the pixelated emitter array. The spherical lenses are configured to deflect or project the light of the assigned pixel onto different areas of the projection surface.

According to at least one embodiment, at least one spherical lens is arranged off-center with respect to at least one of the first and the at least one second pixel as viewed in an emission direction of the pixelated emitter array. By arranging the spherical lenses eccentrically above a pixel, it is possible to generate different deflection angles in order to ensure that the light emitted by the pixels is deflected onto different areas of the projection surface, i.e. “pixelated” onto the projection surface.

According to at least one embodiment, the first and the at least one second pixel are each formed by or comprise a Vertical Cavity Surface Emitting Laser (VCSEL). The VCSELs may comprise, for example, a III-V semiconductor comprising one of the following material systems: InGaAlP, AlGaAs and InGaN. However, the first and at least one second pixel can also be formed by or comprise an LED. In particular, the first and the at least one second pixel are arranged to emit radiation, in particular to emit visible light and/or near-infrared radiation.

According to at least one embodiment, radiation of at least two different wavelength ranges is generated by the at least one pixelated emitter array. The individual pixels of the pixelated emitter array preferably only emit radiation from one of the wavelength ranges, so that at least one separate pixel is present for each wavelength range. The wavelength ranges can partially overlap.

According to at least one embodiment, the first pixel is configured to emit light of a first wavelength and the second pixel is configured to emit light of a second wavelength different from the first wavelength.

According to at least one embodiment, a converter layer is arranged on at least one pixel of the pixelated emitter array, which is configured to at least partially convert the light emitted by the pixel into light of a different wavelength, in particular into broadband light. In such a case, it may be preferable that at least one optical filter is additionally arranged in front of the at least one photodetector in order to detect certain wavelengths of the light emitted by the pixelated emitter array and reflected at the projection surface.

According to at least one embodiment, the vital sensor comprises a first and at least one second pixelated emitter array, wherein the pixels of the first pixelated emitter array are configured to emit light of a first wavelength and the pixels of the at least one second pixelated emitter array are configured to emit light of a second wavelength different from the first wavelength.

According to at least one embodiment, the vital sensor comprises at least two, in particular 4 or more, photodetectors which are arranged symmetrically, in particular along a circular virtual line, around the at least one pixelated emitter array.

According to at least one embodiment, the evaluation unit is configured to apply a different current, in particular a higher current, to at least one of the first and at least one second pixel for measuring a vital parameter, in particular the pulse rate, on the basis of the first reference value in order to determine the reference value. For example, the pixels that are used to measure the vital parameter, i.e. that provide a “good” AC-DC ratio, can be energized at a higher current to measure the vital parameter. This can further increase the measurement accuracy and still reduce the energy consumption compared to energizing all pixels.

Furthermore, a method for measuring a vital parameter, in particular the pulse rate, of a human wearer of a vital sensor, in particular a pulse sensor, comprising the steps:
A method of sequentially emitting pulsed light of a wavelength range by means of a first and at least a second pixel of a pixelated emitter array towards the skin of the human wearer, wherein a light pulse generated by the first pixel is directed to a first region of the skin of the human wearer and a light pulse generated by the second pixel is directed to a second region of the skin of the human wearer different from the first region;
detecting the light emitted by the pixels and reflected from the skin of the human wearer by means of at least one photodetector; and
   determining a first reference value for each pixel on the basis of which at least one of the first and the at least one second pixel is driven to measure the vital parameter.

According to at least one embodiment, the method further comprises determining a second reference value during the measurement of the vital parameter, wherein the first reference value is determined again for each pixel if the second reference value falls below a predefined threshold value. The second reference value can be determined in the same way as the measurement data for determining the pulse rate.

According to at least one embodiment, only the pixels for which the first reference value is greater than or equal to a predefined threshold value are controlled to measure the vital parameter.

According to at least one embodiment, the pixels for which the first reference value exceeds a predefined threshold value are energized at a higher current to measure the vital parameter.

According to at least one embodiment, at least one optical element is arranged between the at least one pixelated emitter array and the skin of the human wearer and is configured to direct the light pulse generated by the first pixel onto the first region of the skin of the human wearer and the light pulse generated by the second pixel onto the second region of the skin of the human wearer.

According to at least a second embodiment, a vital sensor comprises one or more photodetectors. The at least one photodetector is preferably a semiconductor detector, for example a silicon photodiode.

According to at least one embodiment, the vital sensor is formed by a pulse sensor which is configured to determine a vital parameter, in particular the pulse rate, of a human wearer of the vital sensor.

According to at least one embodiment, the vital sensor comprises at least two semiconductor light sources. The semiconductor light sources are preferably light-emitting diodes, LEDs for short, or laser diodes and are set up to emit radiation, in particular to emit visible light and/or near-infrared radiation. Overall, the semiconductor light sources preferably generate radiation of at least two different wavelength ranges. Each of the semiconductor light sources preferably only emits radiation from one of the wavelength ranges, so that at least one separate semiconductor light source is present for each wavelength range. The wavelength ranges can partially overlap.

According to at least one embodiment, the semiconductor light sources are arranged around the at least one photodetector. The semiconductor light sources may all be at the same distance from the at least one photodetector, or there may be different distances between the semiconductor light sources and the at least one photodetector. In this configuration in particular, several photodetectors may be present. The photodetector can be located centrally between the semiconductor light sources. The semiconductor light sources can be located on different sides or all on the same side of the photodetector.

According to at least one embodiment, the vital sensor comprises one or more electronic evaluation units. The at least one evaluation unit is set up to operate the semiconductor light sources in pulsed mode. The semiconductor light sources are temporarily operated sequentially by means of the evaluation unit. This means that, at least occasionally, the semiconductor light sources emit one after the other and not simultaneously. It is also possible for the semiconductor light sources to be operated permanently sequentially and therefore one after the other, so that no two semiconductor light sources are emitted simultaneously at any time.

According to at least one embodiment, the evaluation unit is set up to weight detection signals of the at least one photodetector differently from reflected light of the semiconductor light sources, for example to determine a pulse frequency of a human wearer of the vital sensor. This means that the radiation from at least one of the semiconductor light sources is used for pulse measurement. The at least one semiconductor light source used for pulse measurement thus irradiates the skin and an inner skin layer (layer with a higher density of blood vessels) of the wearer in pulsed form, the radiation is then at least partially reflected by the skin and the inner skin layers and reaches the photodetector. However, only the radiation reflected in the inner skin layers and picked up by the photodetector leads to the detection signals and carries the information about the heart rate of the human wearer of the vital sensor. The radiation reflected from the skin, on the other hand, leads to interference signals.

Different weighting means that the detection signal resulting from the radiation of certain semiconductor light sources is included in the pulse measurement to different degrees at different times. The weighting is determined on the basis of the ratio of interference information to pulse information. If the ratio of interference information to pulse information exceeds a predefined threshold value, in particular if the proportion of interference information outweighs the pulse information, the detection signal resulting from the radiation of certain semiconductor light sources, for example, cannot be used for pulse measurement in a certain time period; this detection signal is therefore rejected by the evaluation unit. Different weighting means that the semiconductor light source whose detection signal would be rejected by the evaluation unit is not operated at all or is operated with a reduced average current in the relevant time period.

In at least one embodiment, the vital sensor comprises at least one photodetector, at least two semiconductor light sources which are set up to emit in different wavelength ranges and/or which are arranged at different distances around the photodetector, and at least one electronic evaluation unit. The semiconductor light sources are set up to be operated in a pulsed and at least time-sequential manner by means of the evaluation unit. Furthermore, the evaluation unit is set up to weight detection signals of the photodetector differently from reflected light of the semiconductor light sources in order to determine a pulse frequency of a human wearer of the vital sensor.

Wearable devices such as heart rate monitors, in-ear headphones or smartphones with optical heart rate sensors often measure the heart rate incorrectly and not reproducibly. The faulty measurements are caused in particular by the fact that conventional sensors usually only have a few LEDs, usually only one or two LEDs, and only one photo diode (PD) with which the measurements are carried out.

The measurements are therefore very sensitive to different skin types, in particular hair growth, fat on the skin surface, skin thickness and skin color. In addition, changes in the position of the sensor have a strong effect on the measurement. Shifts of the sensor by a few millimeters from the previous measurement can make a big difference.

With optical-based pulse sensors, the heartbeat is measured by means of the reflection of light that is coupled into the skin by the LEDs. The ratio of reflected light to light absorbed by the blood depends on the current amount of blood in the upper and/or inner layers of the skin. The penetration depth of the radiation from the semiconductor light sources into the skin is preferably at least 1 mm. The pulse is determined by this change in absorption in the skin.

With conventional sensors, the LEDs for pulse measurement all emit radiation of the same wavelength, whereby other wavelengths can be used for blood oxygen measurement, but are not usually used for pulse determination. In addition, with conventional sensors, all LEDs switch on and off sequentially one after the other at the same time, which makes this type of rigid measurement very prone to error, as a relatively large amount of light always reaches the PD, which is not reflected by the blood but by the skin or scattered back from the upper layers of the skin.

The proportion of light that is absorbed by the blood, or the signal that changes due to the variation in blood volume and is measured by the PD, is usually only in the range of 0.5% to 10% of the total measured signal, in particular only about 1%.

The measurements are preferably carried out at time intervals that are significantly shorter than the pulse duration in order to save energy.

As the measurement data is very susceptible to interference due to the small number of LEDs and the rigid current programming of conventional sensors, these two parameters are modified and/or flexibly configured in the sensor described here to reduce sensitivity to interference and for self-calibration. For this purpose, several LEDs or LED groups are placed around one or more PDs. LEDs with a single emission wavelength can be used, but also LEDs with different emission wavelengths.

Specific light colors can be emitted from the LED groups with more than one LED with different wavelengths. Furthermore, a driver is integrated into the evaluation unit and thus into the heartbeat sensor, so that each individual LED can be driven with a specific current defined by the calibration.

Calibration is carried out as follows, for example: The LEDs or LED groups are controlled individually or in groups. A signal modulation is evaluated on the PD that corresponds to the respective LED or LED group. For the subsequent measurements of the pulse, the LEDs or LED groups with the maximum signal modulation are evaluated higher and/or operated with a higher current.

After a certain number of pulse measurement cycles, for example after at least 10 and/or after a maximum of 100 cycles or a maximum of 300 cycles or a maximum of 1000 cycles, the evaluation unit controls the drivers and thus the semiconductor light sources again for a new calibration measurement. This calibration measurement is then used again for the next pulse measurement cycles. During calibration, the optimum spectrum and the LEDS or LED groups that lead to the best detection signal are recorded. This reduces interference, particularly due to the position and orientation of the vital sensor.

The data can be analyzed using a neural network. The evaluations of the various measurement data are then based on the previous measured values. The sensor then adapts independently to changing measurement situations.

The respective calibration greatly reduces changing interferences resulting from different skin types, as the subsequent measurements are always configured to the current skin surface, for example the hair growth at the current location, a layer of fat, thickness, skin color and sweat.

Furthermore, the sensor preferably determines the current position of the LEDs and the PD in relation to the skin surface during calibration. In this way, the proportion of LEDS or LED groups that are far away from the skin surface in the relevant time period and whose light is therefore poorly coupled into deeper skin layers can be kept very low during calibration. Preferably, only those LEDS or LED groups are operated that efficiently couple their radiation into the skin. This ensures that as little stray light as possible reaches the PD. The proportion of light that reaches deeper layers of the skin is greater in relation to the total amount of light.

According to at least one embodiment, the semiconductor light sources and/or the groups of semiconductor light sources are optically separated from each other. This means that there is no direct line of sight between the semiconductor light sources and/or the groups of semiconductor light sources.

According to at least one embodiment, the semiconductor light sources are formed by micro-LEDs. This allows a greater wavelength variance to be achieved, i.e. a greater number of different wavelength ranges can be present.

According to at least one embodiment, different photodetectors are used. The photodetectors preferably have different distances to the LEDs or to the LED groups.

The pulse can be determined efficiently using various wavelengths in the spectral range from 500 nm to 600 nm, in particular in the green spectral range. Additional parameters such as blood pressure, oxygen saturation, haemoglobin and/or skin moisture can also be determined using other wavelengths, particularly in the red and near-infrared spectral range.

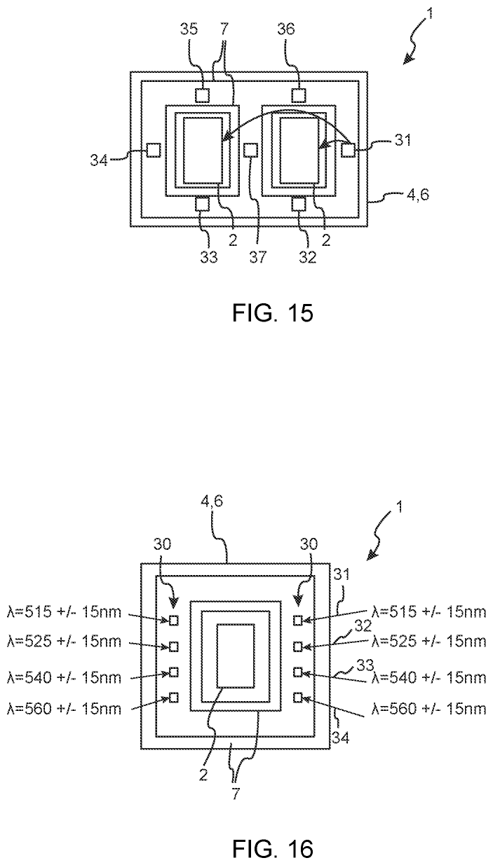

Pulse oximetry is described, for example, in publication WO 2018/206391 A1, see in particular FIG. 15 and page 21, last paragraph to page 22, third paragraph. This disclosure is incorporated by reference.

According to at least one embodiment, the vital sensor comprises at least four of the semiconductor light sources which have different emission wavelengths from one another, preferably in the range from 500 nm to 600 nm inclusive.

According to at least one embodiment, the semiconductor light sources are arranged symmetrically around the at least one single-channel photodetector. Alternatively, there is an asymmetrical arrangement.

According to at least one embodiment, an average distance between the at least one photodetector and the semiconductor light sources is at least 0.5 mm or 1 mm or 2 mm. Alternatively or additionally, this distance is at most 6 mm or 5 mm or 4 mm.

According to at least one embodiment, the semiconductor light sources are at different distances from a center of the at least one photodetector. This applies in particular in the top view of a detection surface of the photodetector.

In addition, a method for measuring a vital parameter, in particular pulse rate, is disclosed. The method is performed with a vital sensor as described in connection with one or more of the above embodiments. Features of the vital sensor are therefore also disclosed for the method and vice versa.

In at least one embodiment, the method comprises the following steps, preferably in the order indicated:
Attach the vital sensor to the carrier,
pulsed and at least time-sequential operation of the semiconductor light sources, and
Determining the pulse rate of the wearer,
   whereby the detector signals on the photodetector are weighted differently when determining the pulse frequency and a weighting of the detector signals changes over time, whereby the detector signals result from light from the semiconductor light sources reflected in the skin of the wearer.

According to at least one embodiment, the semiconductor light sources are operated in pulsed mode at a frequency of at least 20 Hz or 30 Hz or 50 Hz. Alternatively or additionally, this frequency is at most 500 Hz or 300 Hz or 150 Hz. Preferably, this frequency is around 100 Hz, for example between 80 Hz and 120 Hz.

According to at least one embodiment, a pulse duration of the radiation emitted by the semiconductor light sources is at least 1 us or 5 us or 30 μs. Alternatively or additionally, the pulse duration is at most 2 ms or 0.3 ms or 0.15 ms.

According to at least one embodiment, the weighting of the detector signals is checked and/or changed at a repetition rate between 0.3 Hz and 50 Hz inclusive or between 0.5 Hz and 30 Hz inclusive or between 2 Hz and 20 Hz inclusive.

Alternatively, the weighting is checked and/or changed at a lower frequency than the pulse frequency. For example, the weighting is checked over at least two pulse cycles. In this case, the repetition rate is between 0.01 Hz and 0.5 Hz. The duration of the check is then in particular between 0.4 s and 4 s inclusive.

According to at least one embodiment, a subgroup of the semiconductor light sources is permanently weighted more heavily than the other semiconductor light sources, depending on the skin type of the wearer. For example, the semiconductor light sources are grouped into groups of the same emission wavelengths. The evaluation unit can comprise a memory unit for this purpose in particular.

According to at least one embodiment, the vital sensor was or is trained by means of neural learning. This means that the accuracy of the vital sensor can be continuously improved based on the ongoing measurements. This is possible, for example, in comparison with other pulse measuring devices such as treadmills or bicycles.

In the following, an optoelectronic vital sensor described here and a method described here are explained in more detail with reference to the drawing using examples of embodiments. Identical reference signs indicate identical elements in the individual figures. However, no references are shown to scale; rather, individual elements may be shown in exaggerated size for better understanding.

It shows:

FIG. 1 a schematic representation of a photocurrent detected by a photodetector during a measurement of the pulse rate of a human being;

FIG. 2 a schematic representation of the distribution of arteries on the wrist of a human being;

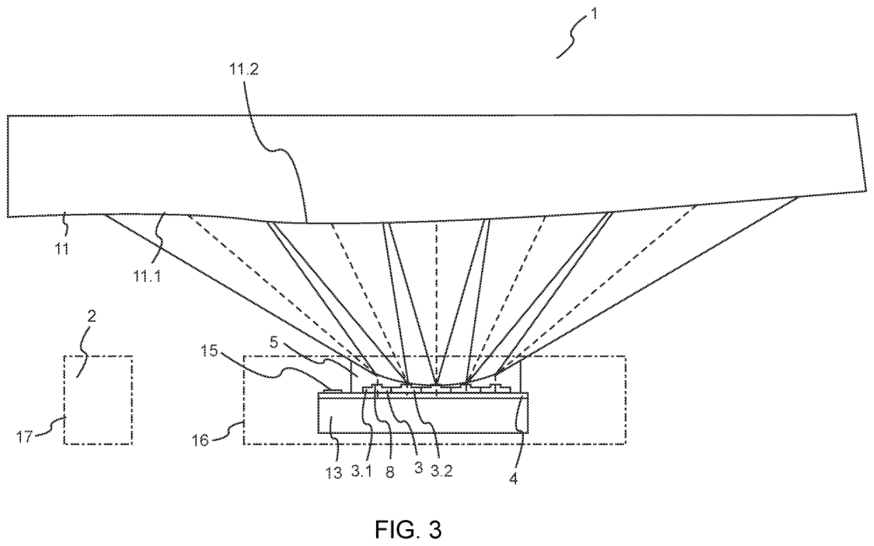

FIG. 3 a sectional view of a vital sensor according to some aspects of the proposed principle;

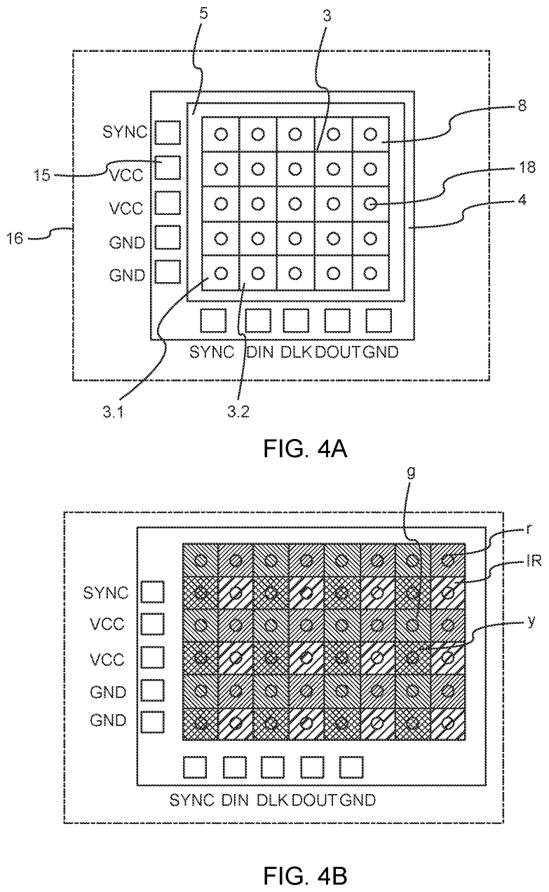

FIGS. 4A and 4B each a top view of a pixelated emitter array according to some aspects of the proposed principle;

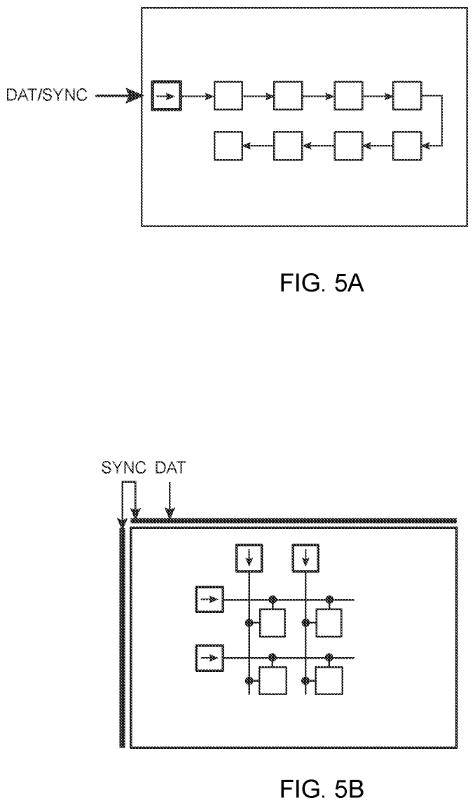

FIGS. 5A and 5B each a schematic circuit diagram of a pixelated emitter array according to some aspects of the proposed principle;

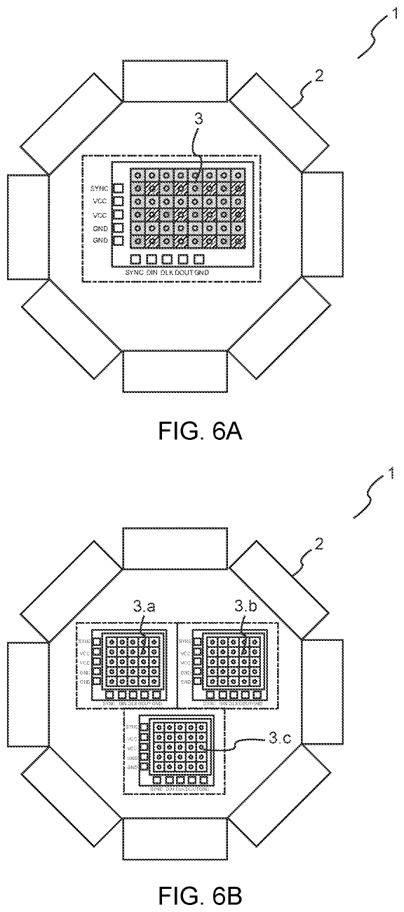

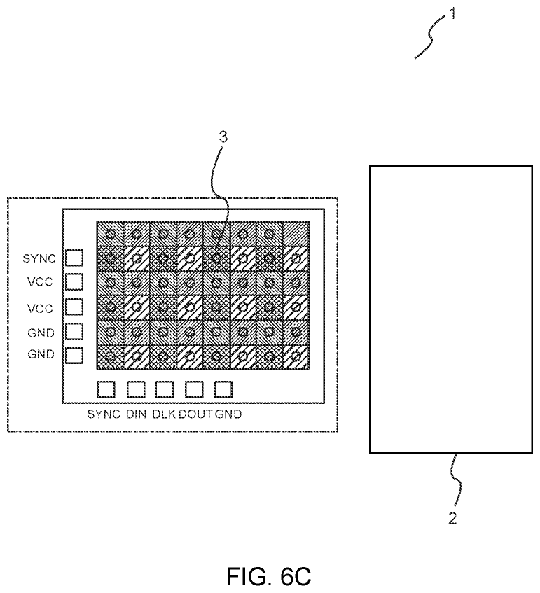

FIGS. 6A to 6C each a top view of a vital sensor according to some aspects of the proposed principle;

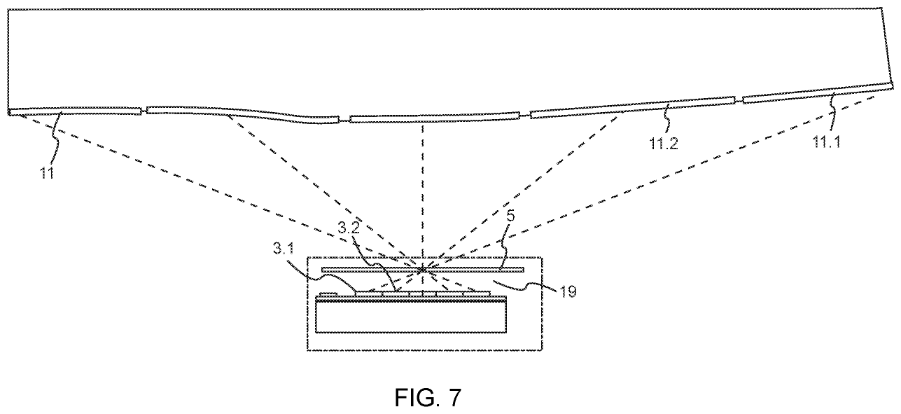

FIG. 7 a sectional view of a vital sensor according to some aspects of the proposed principle;

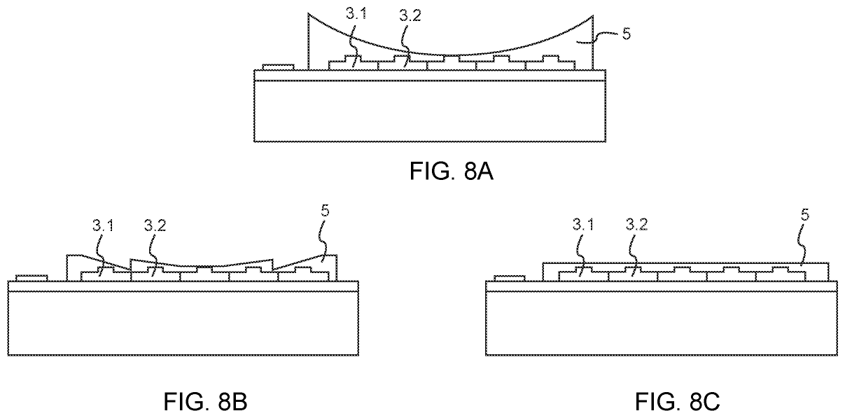

FIGS. 8A to 8C each a sectional view of a pixelated emitter array with an optical element arranged thereon according to some aspects of the proposed principle;

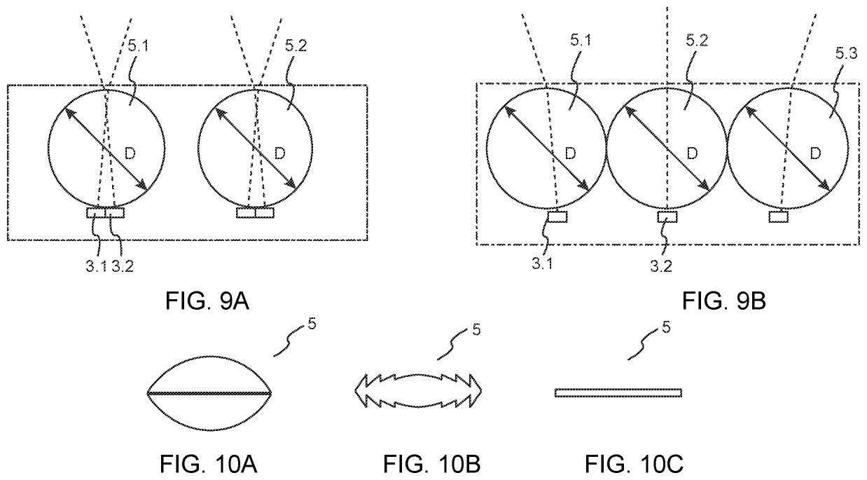

FIGS. 9A and 9B each a schematic sectional view of an optical element according to some aspects of the proposed principle;

FIGS. 10A to 10C exemplary embodiments of optical elements according to some aspects of the proposed principle;

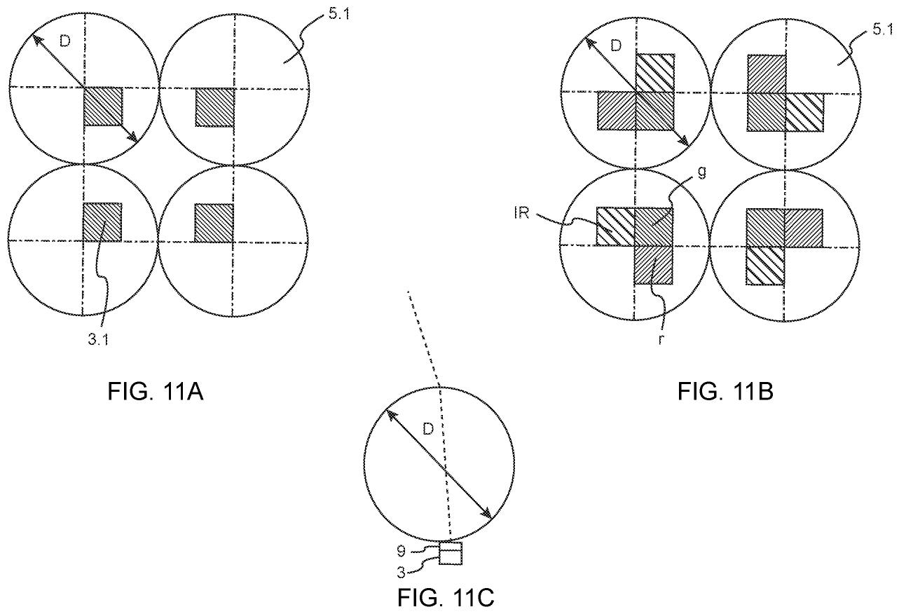

FIGS. 11A and 11B each a top view of an optical element with pixels arranged thereunder according to some aspects of the proposed principle;

FIG. 11C a sectional view of an optical element with a pixel arranged underneath according to some aspects of the proposed principle;

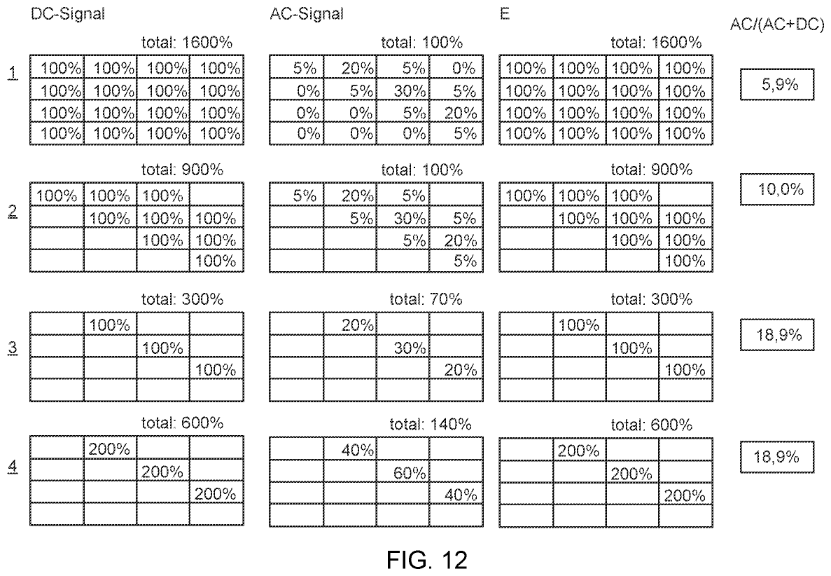

FIG. 12 a schematic representation of an exemplary calculation for a pixelated emitter array for measuring a vital parameter according to some aspects of the proposed principle;

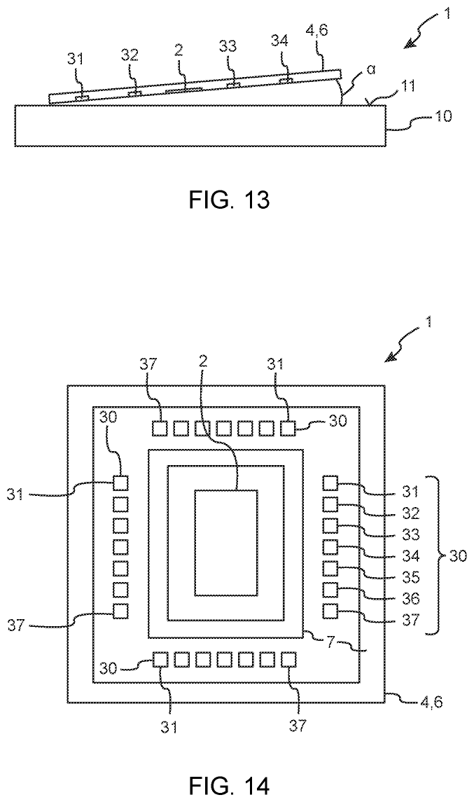

FIG. 13 a schematic sectional view of an embodiment of a vital sensor described herein on a skin of a wearer; and

FIGS. 14 to 19 schematic top views of embodiments of the vital sensors described here.

FIG. 2 shows a schematic representation of the distribution of arteries in the wrist of a human being. It can be seen from the figure that the arteries in the hand can be both very wide and fine, but in particular are very branched. The density of the arteries in different areas of the wrist varies. The figure shows approximately the position of a wristwatch 12. Within this area 12 there are places with low 12.1, medium 12.2 and high 12.3 arterial density.

FIG. 3 shows a sectional view of a first embodiment of a vital sensor 1 according to some aspects of the proposed principle. The vital sensor comprises a pixelated emitter array 3 with a first pixel 3.1 and at least one second pixel 3.2. In the case shown, the pixelated emitter array 3 comprises at least five pixels of VCSEL emitters, which can be seen in the sectional view, but the pixelated emitter array 3 may have further pixels perpendicular to the plane of the drawing. The pixelated emitter array 3 is arranged on a substrate 13, for example a silicon substrate, with an active matrix control electronics (IC) 4 or evaluation unit. The control electronics or evaluation unit is configured to control the first 3.1 and the at least one second pixel 3.2 in a pulsed and time-sequential manner, in particular to determine a first reference value, or to control at least one of the first and the at least one second pixel simultaneously to determine a second reference value. Each emitter corresponds to a pixel and can be controlled individually via the control electronics. An aperture 8 is arranged above each of the individual emitters, which is configured to limit the cross-section of the light emitted by the pixels, in particular to one light spot. Power and data are supplied to the IC or the VCSEL emitters via bond pads 15.

The control electronics can be connected to the VCSEL emitters via an electrical and mechanical interconnect, for example. The interconnect can be made using a wafer-to-wafer, chip-to-wafer or chip-to-chip process. The pixelated emitter array on the substrate (imager chip) sits in an emitter housing 16 which is not specified further. The function of the emitter housing is the electrical and thermal connection to a circuit board or similar (e.g. as an SMT component: QFN, premolded, ceramic, PCB, . . . ). It also protects the imager chip from environmental influences (encapsulation, e.g. epoxy, silicone) and ensures efficient light extraction (e.g. with TiO2 reflector). However, the emitter housing is optional. The imager chip can also be mounted directly on a circuit board or similar (Chip on Board CoB).

The laser light emitted by the pixels, in particular slightly divergent laser light, is deflected by an optical element 5, in the illustrated case in the form of concave optics, and illuminates a projection surface, in particular the skin of a human wearer of the vital sensor, in separate areas. Specifically, the light emitted by the first pixel 3.1 and focused by an aperture 8 is deflected by the optical element onto a first region of the skin 11.1 and the light emitted by the second pixel 3.2 and focused by an aperture 8 is deflected by the optical element onto a second region of the skin 11.2 that is different from the first and illuminates it in each case. The laser light penetrates the skin in the respective areas and is reflected there. The reflected light is received by a photodetector 2, which is located next to or slightly above or below the pixelated emitter array 3.

The photodetector 2 sits in a detector housing 17 that is not specified further. The function of the detector housing is the electrical and thermal connection to a circuit board or similar (e.g. as an SMT component: QFN, premolded, ceramic, PCB, . . . ). It also protects the photodetector 2 from environmental influences (encapsulation, e.g. epoxy, silicone) and ensures efficient light coupling (e.g. with TiO2 reflector). However, the detector housing is optional. The photodetector 2 can also be mounted directly on a circuit board or similar (chip on board CoB). The detector housing 17 and emitter housing 16 can also be a system housing.

FIGS. 4A and 4B each show a top view of a pixelated emitter array according to some aspects of the proposed principle. In the top view, the aperture 8 assigned to each pixel can be seen, through which the cross-section of the light emitted by the pixels is limited to a point of light. Due to this arrangement, it can also be referred to below as a so-called VCSEL eye 18, through which the pixels emit light. For the electrical contacting of the pixelated emitter array, there are connections or bond pads 15 for the power supply (VCC, GND) and for data (DIN, DOUT, CLK, SYNC) on the control electronics or evaluation unit 4.

The pixelated emitter array 3 or the pixels of a pixelated emitter array can be configured to emit light of the same wavelength, as shown in FIG. 4A, but the pixelated emitter array 3 can also have pixels that emit light of different wavelengths, as shown in FIG. 4B. In the example shown in FIG. 4B, the pixelated emitter array 3 can have pixels that emit light of the colors green g, red r and yellow y, for example. In addition, the pixelated emitter array can have 3 pixels that emit infrared light IR. Green, red and infrared light are in particular the colors commonly used today to determine a vital parameter; yellow light can also be used to obtain even more information.

FIGS. 5A and 5B each show a schematic circuit diagram of a pixelated emitter array according to some aspects of the proposed principle. The driver electronics in the control electronics can be implemented in the form of daisy-chain programming, as shown in FIG. 5A, or as cross-matrix programming, as shown in FIG. 5B. With daisy-chain programming, data for controlling the pixels of the pixelated emitter array is supplied serially to the control electronics and stored in each pixel after the complete chain of pixels has been run through. With a cross matrix, on the other hand, the data for controlling the pixels of the pixelated emitter array is programmed line by line and stored in the pixels. Both types of control ensure that it is possible to determine externally which pixels are to be energized and which are not. FIGS. 6A to 6C show embodiments of a vital sensor according to some aspects of the proposed principle in plan view. As shown in FIG. 6A, several photodetectors 2 can be arranged in a ring around a pixelated emitter array 3. In the illustrated case, eight photodetectors 2 are arranged around a pixelated emitter array 3, but both less than eight and more than eight photodetectors 2 may be arranged pixelated emitter array 3. However, the photodetectors should be arranged symmetrically and, in particular, at the same distance from the pixelated emitter array 3 around the pixelated emitter array 3.

As shown in FIG. 6B, however, several pixelated emitter arrays 3.a, 3.b, 3.c can also be arranged within a ring of photodetectors 2. In the case of several pixelated emitter arrays, for example, each pixelated emitter array is configured to emit light of a different wavelength, whereas in the case of only one pixelated emitter array, this can be configured to emit light of different wavelengths. It is also conceivable that, as shown in FIG. 6C, only one photodetector 2 is assigned to a pixelated emitter array 3.

In particular, however, a light trap or optical separation may be provided between the photodetectors and the emitters or pixels of the pixelated emitter arrays in order to avoid direct radiation without measurement information, so-called crosstalk.

FIG. 7 shows a side view of an embodiment of a vital sensor similar to FIG. 3. The optical element 5 is formed by refractive or flat optics, but the pixels of the emitter array are each formed by LEDs. Due to the Lambertian radiation behavior of LEDs, pixelated imaging of the light emitted by the LEDs onto the skin with refractive or flat optics is significantly more difficult compared to VCSELs. An example calculation was used to determine which lens radius would be necessary for a given pixelated emitter array in order to enable pixelated imaging of the individual light points on the skin. If we consider the lens laws for imaging the pixelated emitter array on the skin with a magnification scale of 10:1 (illuminated area pixel:illuminated area on the skin), we obtain a lens radius of approx. 0.15 mm for an object width 19 of 0.3 mm (distance between lens and emitter array). This would have to be accommodated on an object width of 0.3 mm and at the same time image the entire chip surface. Although this is difficult to achieve with refractive optics, it is certainly possible.

FIGS. 8A to 8C each show a sectional view of a pixelated emitter array 3 with an optical element 5 arranged thereon according to some aspects of the proposed principle. FIG. 8A shows a refractive lens arranged on the emitter array. FIG. 8B shows a Fresnel step lens arranged on the emitter array and FIG. 8C shows a diffractive lens or a lens made of metamaterial arranged on the emitter array, whereby a structuring thereof is not shown in the figure. The aim of the optical element 5 is to have the smallest possible height. A refractive, concave lens fulfills these requirements well for an emitter array with VCSELs. Due to its height, however, it can also make sense to design the optical element 5 as a Fresnel stepped lens or as a diffractive lens or lens made of metamaterial.

FIGS. 9A and 9B each show a schematic sectional view of one or more optical elements 5 according to some aspects of the proposed principle. In each case, the optical element 5 is configured such that it comprises one or more spherical lenses 5.1, 5.2, 5.3, which are each arranged above one or more pixels 3.1, 3.2 of the emitter array 3.

The previous example calculation was used to determine how the spherical lens(es) would have to be arranged over one or more pixels of the emitter array 3 in order to enable a pixelated image of the individual light points on the skin and at the same time a low overall height. The calculated lens radius of 0.15 mm can be easily realized using a spherical lens with a diameter D of 0.3 mm. However, with a large beam angle of the pixels in combination with the relatively small spherical lens, no more than 2×2 pixels per lens can be imaged without losing a lot of light next to the lens. In order to achieve the desired beam deflection on the skin, the two pixels 3.1 and 3.2 per spherical lens 5.1 shown in FIG. 9A are arranged outside the optical axis of the sphere. The magnification is again selected to be approximately 10:1, which results in a 0.5 mm illuminated spot on the skin with a luminous area of 50 μm. In order to illuminate the skin with more than 2×2 pixels, several spherical lenses can be placed next to each other as shown in FIG. 9A.

To further improve the image, a spherical lens can be assigned to each pixel, as shown in FIG. 9B. In order to achieve the desired deflection onto the skin, the emitters are arranged outside the optical axis of the sphere, except for the center emitter. In this case, it is also possible that there is no pixelated emitter array in which all emitters are located on a common driver IC; instead, the emitters can be arranged at a distance from each other on a substrate and can be controlled separately, thus forming the emitter array.

To reduce the thickness of the structure, the lens shapes shown in FIGS. 10a to 10C can be used instead of spheres, such as thick lenses (FIG. 10A), Fresnel step lenses (FIG. 10B) or diffractive lenses (FIG. 10C), and can be used over one pixel at a time.

FIGS. 11A and 11B each show a top view of an optical element with pixels arranged underneath according to some aspects of the proposed principle. In the example shown in FIG. 11A, 2×2, i.e. four pixels are arranged under four spherical lenses, i.e. one spherical lens per pixel. However, it is also conceivable to have 2×3, 3×3, 4×4, 3×4, 2×4, etc. pixels under a corresponding number of spherical lenses. The distance between the emitters and the optical element, the distance between the optical element and the projection surface, the aperture, the sphere radius and also the desired optical design determine how far the emission surface is from the optical axis of the respective spherical lens.

As shown in FIG. 11B, several emitters, in particular emitters emitting different colors, can also be arranged under a spherical lens. In the present case, one pixel or emitter emitting red light r, one emitting green light g and one emitting infrared light IR are arranged.

FIG. 11C a sectional view of an optical element with a pixel arranged thereunder according to some aspects of the proposed principle. A converter layer 9 is arranged on the pixel, which is configured to at least partially convert the light emitted by the pixel into light of a different wavelength, in particular into broadband light. In such a case, it may be preferable that at least one optical filter is additionally arranged in front of the at least one photodetector in order to detect certain wavelengths of the light emitted by the pixelated emitter array and reflected at the projection surface. This can apply in particular in the event that the wavelength multiplexing is not carried out via sequential operation of the emitters or pixels, but via multiplexing at the detector (e.g. spectrometer or photodetector with different spectrally sensitive areas). On a blue LED, for example, there may be a light converter that generates green, yellow, red and near and far infrared light from the blue light via various phosphors.

FIG. 12 shows a schematic representation of an exemplary calculation for a pixelated emitter array for measuring a vital parameter on the basis of the first reference value measured for each pixel. The starting point is a pixelated emitter array with 4×4 pixels, which is used to illuminate the skin on a person's wrist. In the left column, the DC component of the signal measured at the photodetector is assumed to be 100% due to the light reflected at the skin from each of the 4×4 pixels. The AC signal in the middle column is additionally superimposed on the DC component (as also described in FIG. 1). In this example, areas with up to 30% AC component were arbitrarily assumed, which are measured in different areas on the skin, depending on the arterial density.

In example 1 (top line), it is assumed that all emitter pixels light up 100% during the measurement. The energy E required for this is therefore calculated as 1600%. The signal detected at the photodetector is 1600% (DC signal)+100% (AC signal) according to the tables. The AC-DC calculated ratio for this is 100%/(1600%+100%)≈5.9%.

In example 2 (second line), only areas on the skin that contain an AC signal are illuminated. The signal detected at the photodetector drops significantly from 1700% to only 900% (DC signal)+100% (AC signal). However, the AC-DC ratio calculated for this increases significantly to 100%/(900%+100%)=10% and the energy consumption E decreases significantly from 1600% to 900%, as only nine pixels of the emitter array need to be energized at 100% compared to 16 pixels.

In example 3 (third line), only the areas on the skin with the highest AC signal are now illuminated. The trend from example 2 continues clearly. The signal detected at the photodetector drops from 900% to only 300% (DC signal)+70% (AC signal). However, the AC-DC ratio calculated for this increases significantly to 70%/(300%+70%)≈18.9% and the energy consumption E drops significantly from 900% to 300%, as only three pixels of the emitter array need to be energized at 100% compared to nine pixels.

In example 4 (last line), the AC signal that fell to 70% in example 3 is to be raised again (to 140%). This is achieved by redirecting more illuminance to the areas with the highest signal component. For example, this can be achieved by applying a higher current to the corresponding pixels, for example twice as high. As a result, the signal-to-noise ratio or the AC/DC signal ratio can be improved, at least compared to examples 1 and 2, and kept at least at the same level compared to example 3. The absolute AC signal can thus even be improved compared to all three examples, and the energy consumption can be significantly improved compared to example 1 and also compared to example 2.

FIG. 13 shows an embodiment of a vital sensor, in particular pulse sensor 1, and its mode of operation is explained in more detail. The pulse sensor 1 comprises several semiconductor light sources 31, 32, 33, 34, which are integrated together with a photodetector 2 on a substrate 6. The substrate 6 also contains an evaluation unit 4 for controlling the semiconductor light sources 31, 32, 33, 34 and the photodetector 2 and for signal evaluation.

The evaluation unit 4 is an IC, in particular an ASIC. The photodetector 2 is, for example, a Si photodiode, PD for short. The semiconductor light sources 31, 32, 33, 34 are preferably LEDs which display different wavelengths of maximum emission intensity. The wavelengths of maximum emission intensity are preferably in the wavelength range from 500 nm to 600 nm.

It is possible that the semiconductor light sources 31, 32, 33, 34 are used exclusively for pulse measurement. Alternatively, the semiconductor light sources 31, 32, 33, 34 can also be used to measure other biometric variables.

The heart rate sensor 1 is integrated into a heart rate monitor or a smartwatch, for example. This means that the pulse sensor 1 is in contact with the skin surface 11 of a human wearer 10.

Due to vibrations or changes in position, for example when the wearer 10 is walking, and/or due to hair or local color changes on the skin surface 11 as well as due to deposits such as grease or sweat, the quality of the light coupling of radiation from the semiconductor light sources 31, 32, 33, 34 into the skin varies and thus the signal quality at the photodetector 2, which detects light scattered back from the skin and is used in particular for pulse measurement. This means that the pulse sensor 1, which does not lie flat on the skin surface 11, reflects light directly from the skin surface 11, which leads to a relatively large amount of interfering light on the PD 2.

A calibration frequency for calibrating the pulse sensor 1 is preferably significantly lower than the pulse frequency. For example, the calibration takes at least 2 periods and/or at most 10 periods of the pulse, i.e. heartbeats, in order to select the optimum semiconductor light source 31, 32, 33, 34, which is then used to determine the pulse. Preferably, this also applies to all other embodiments.

During calibration, the semiconductor light sources 31, 32, 33, 34 are operated sequentially and pulsed, for example with one or more pulses with pulse durations of around 100 μs. The photodetector 2 is used to determine which semiconductor light sources 31, 32, 33, 34 contribute how much to a signal. Until the next calibration, the signal contributions at the photodetector 2 of the semiconductor light sources 31, 32, 33, 34, which also emit sequentially during the pulse measurement, are weighted accordingly. If certain semiconductor light sources 31, 32, 33, 34 do not provide a meaningful signal, these semiconductor light sources 31, 32, 33, 34 can also be switched off until the next calibration.

The information content of certain LEDs 31, 32, 33, 34 is therefore rated as very low by an evaluation in the evaluation unit 4. In FIG. 13, this applies to LED 34, which is relatively far away from the carrier 10 due to a temporary tilt angle α between the skin surface 11 and the substrate 6. On the other hand, the light component of LED 31, which is close to the skin surface 11, is rated highly. The current position of the semiconductor light sources 31, 32, 33, 34 is therefore recorded during calibration and taken into account for the pulse measurement.

FIG. 14 shows a further design example. Four groups 30, each with seven linearly arranged semiconductor light sources 31, 32, 33, 34, 35, 36, 37, are arranged around the central photodetector 2. The semiconductor light sources 31, 32, 33, 34, 35, 36, 37 are LEDs with spectral half-widths between preferably 10 nm and 30 nm.

Emission wavelengths of maximum intensity are each between 500 nm and 600 nm. The emission wavelengths of maximum intensity of the semiconductor light sources 31, 32, 33, 34, 35, 36, 37 preferably differ in pairs by at least 5 nm and/or by at most 25 nm. For example, the emission wavelengths of maximum intensity in the groups 30 increase from the semiconductor light source 31 towards the semiconductor light source 37.

Optical barriers 7 are preferably located between the groups 30 and the photodetector 2 as well as in the vicinity of the pulse sensor 1. The barriers 7 are shaped in the form of a frame or ring, for example. External interfering light can be reduced by such barriers 7. In addition, there is no direct line of sight between the photodetector 2 and the semiconductor light sources 31, 32, 33, 34, 35, 36, 37.

The barriers 7 can be formed by projections on the substrate 6 and/or by the semiconductor light sources 31, 32, 33, 34, 35, 36, 37 and the photodetector 2 being recessed in the substrate 6, as shown in FIG. 13. The barriers 7 and/or the substrate 6 are, for example, made of a metal, a plastic such as the printed circuit board material FR4, a particularly colored glass and/or a ceramic.

In addition to position control, as explained in connection with FIG. 13, the pulse sensor 1 of FIG. 14 can also be calibrated in terms of spectral properties. In this way, the semiconductor light sources 31, 32, 33, 34, 35, 36, 37 whose emission spectrum provides the best signal at the photodetector 2 can be used for pulse measurement.

This means that calibration can be performed with regard to both the position and the emission spectrum of the semiconductor light sources 31, 32, 33, 34, 35, 36, 37. Calibration to the emission spectrum makes it possible in particular to optimize the color of the skin surface 11.

The semiconductor light sources 31, 32, 33, 34, 35, 36, 37 can be formed by micro-LEDs. Viewed from above, the dimensions of the semiconductor light sources 31, 32, 33, 34, 35, 36, 37 are then preferably at most 0.2×0.2 mm2. If no micro-LEDs are used, the dimensions of the semiconductor light sources 31, 32, 33, 34, 35, 36, 37 are, for example, 0.5×0.5 mm2. The semiconductor light sources 31, 32, 33, 34, 35, 36, 37 can generate the desired emission spectrum directly from a semiconductor layer sequence. Alternatively, color filters and/or phosphors can be used to obtain the desired emission spectrum from light of a semiconductor layer sequence.

In FIG. 15, two of the photodetectors 2 are present, around and between which the semiconductor light sources 31, 32, 33, 34, 35, 36, 37 are arranged. Again, each photodetector 2 has a four-sided geometry, with the semiconductor light source 37 located between the two photodetectors 2. The semiconductor light sources 31, 32, 33, 34, 35, 36, 37 all emit the same wavelength spectrum within the manufacturing tolerances. Calibration is used to determine which of the semiconductor light sources 31, 32, 33, 34, 35, 36, 37 is best suited for pulse measurement. In other words, the calibration in this case is aimed at the position of the semiconductor light sources 31, 32, 33, 34, 35, 36, 37 and not at the wavelength range.

The configuration of FIG. 15, according to which a position of the semiconductor light sources 31, 32, 33, 34, 35, 36, 37 is decisive for the calibration, can be combined with a calibration based on the emission spectrum. For example, the configurations of FIGS. 14 and 15 can be combined with each other. The same applies to the other embodiments.

According to FIG. 16, the two groups 30 are arranged on both sides of the photodetector 2. The emission wavelengths of maximum intensity of the semiconductor light sources 31, 32, 33, 34 are indicated in FIG. 16. These emission wavelengths preferably also apply in all other embodiments.

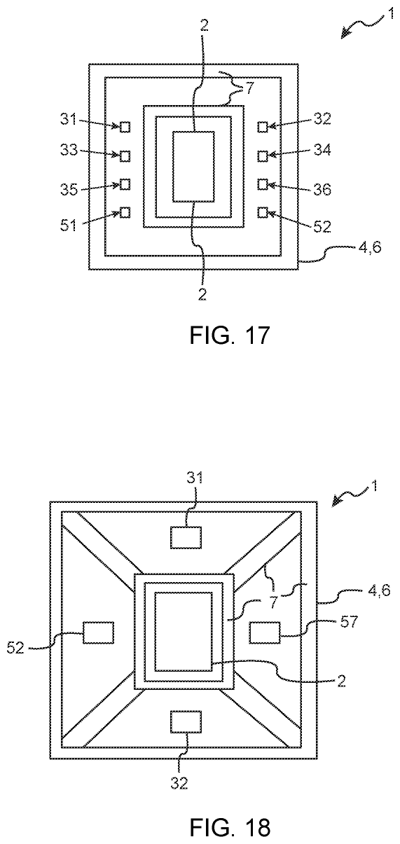

FIG. 17 illustrates that further light sources 51, 52 are present. The additional light sources 51, 52 are preferably also LEDs. For example, the additional light sources 51, 52 emit near-infrared radiation or red light in order to additionally enable pulse oximetry.

For example, the semiconductor light sources 31, 32 emit green light, the semiconductor light sources 33, 34 emit yellow light, the semiconductor light sources 35, 36 emit red light and the other light sources 51, 52 emit near-infrared radiation.

In the embodiment example of FIG. 18, the light sources 31, 32, 51, 52 are arranged in a cross shape around the photodetector 2. For example, the semiconductor light sources 31, 32 generate blue and green light, and the other light sources 51, 52 generate orange and red light.

The barriers 7 are X-shaped and frame-shaped, so that the barriers 7 also extend between neighboring light sources 31, 32, 51, 52. Such a configuration is also possible in all other embodiment examples.

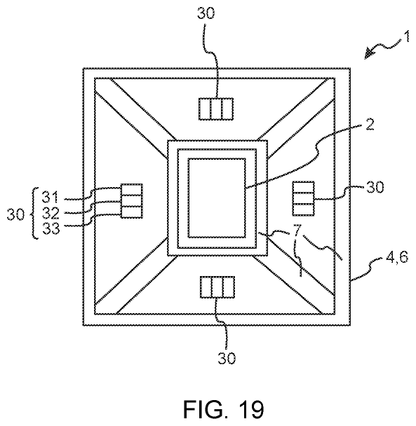

According to FIG. 19, the groups 30 are arranged like the light sources 31, 32, 51, 52 in FIG. 18. The groups 30 are structured similarly to pixels. For example, the groups 30 are RGB units each with a red emitting semiconductor light source 33, a green emitting semiconductor light source 32 and a blue emitting semiconductor light source 31.

With the different wavelengths, an optimum emission spectrum for the skin type of the wearer of the pulse sensor 1 can be determined during calibration and precisely set and/or stored for subsequent measurements.

The invention described herein is not limited by the description based on the embodiments. Rather, the invention includes any new feature as well as any combination of features, which includes in particular any combination of features in the patent claims, even if this feature or combination itself is not explicitly stated in the patent claims or embodiments.

In the following, embodiments of a pulse sensor and a method for operating a pulse sensor as Objects are listed again by way of example. The following objects present various aspects and embodiments of the proposed principles and concepts, which can be combined in various ways. Such combinations are not limited to those indicated below:
1. Vital sensor, in particular pulse sensor (1) with
   at least one photodetector (2),
   at least two semiconductor light sources (31 . . . 37) which are set up for emission in different wavelength ranges and/or which are arranged at different distances around the photodetector (2),
   at least one electronic evaluation unit (4),
   wherein
   the semiconductor light sources (31 . . . 37) are configured to be operated in a pulsed and at least time-sequential manner by means of the evaluation unit (4),
   the evaluation unit (4) is configured to weight detection signals of the photodetector (2) differently from reflected light of the semiconductor light sources (31 . . . 37) in order to determine a vital parameter, in particular the pulse rate, of a human wearer (10) of the vital sensor (1).
2. The vital sensor (1) according to the previous item, comprising at least four semiconductor light sources (31 . . . 37) having different emission wavelengths from each other in the range from 500 nm to 600 nm inclusive,
   wherein the semiconductor light sources (31 . . . 37) are arranged symmetrically around the at least one single-channel photodetector (2).
3. The vital sensor (1) according to item 1,
   wherein all semiconductor light sources (31 . . . 37) have the same emission wavelength,
   wherein at least two of the semiconductor light sources (31 . . . 37) have different distances from one another from the at least one photodetector (2).
4. The vital sensor (1) according to any one of the preceding items,
   comprising at least two photodetectors (2),
   wherein between any of the photodetectors (2) and any of the semiconductor light sources (31 . . . 37) there is no direct line of sight.
5. The vital sensor (1) according to any one of the preceding items,
   comprising at least one further light source (51, 52) with a further emission wavelength range, which is set up for determining a blood oxygen content of the wearer (10).
6. The vital sensor (1) according to any one of the preceding items,
   wherein an average distance between the at least one photodetector (2) and the semiconductor light sources (31 . . . 37) is between 1 mm and 5 mm inclusive,
   wherein different distances of the semiconductor light sources (31 . . . 37) to a center of the at least one photodetector (2) are present.
7. Method, by which the vital sensor (1) according to one of the preceding items is operated, comprising the steps:
   Attaching the vital sensor (1) to the carrier (10),
   pulsed and least time-sequential at operating the semiconductor light sources (31 . . . 37), and
   Determining the wearer's vital signs (10),
   wherein the detector signals at the photodetector (2) are weighted differently when determining the vital parameter and the detector signals result from light of the semiconductor light sources (31 . . . 37) reflected in the skin of the wearer (10), and
   wherein a weighting of the detector signals changes over time.
8. The method according to the preceding item,
   wherein the semiconductor light sources (31 . . . 37) are operated in pulsed mode at a frequency between 30 Hz and 300 Hz inclusive,
   wherein a pulse duration is between 5 us and 0.3 ms inclusive in each case, and
   wherein the weighting of the detector signals is checked and/or changed at a repetition rate of between 0.01 Hz and 0.5 Hz.
9. Method according to one of the two preceding items,
   wherein, depending on a skin type of the wearer (10), a subgroup of the semiconductor light sources (31 . . . 37) is permanently weighted more strongly than the other semiconductor light sources (31 . . . 37).
10. Method according to any one of the three preceding items,
   wherein the vital sensor (1) has been or is being trained by means of neural learning.

### LIST OF REFERENCE SYMBOLS

1 Vital sensor, pulse sensor
10 Carrier of the pulse sensor
11 Projection surface, skin surface
11.1 First region
11.2 Second region
2 Photodetector
3 pixelated emitter array
3.1 first pixel
3.2 second pixel
3.a first emitter array
3.b second emitter array
3.c third emitter array
30 Group of semiconductor light sources
31 . . . 37 Semiconductor light source
4 electronic evaluation unit, control electronics
5 optical element
5.1 spherical lens
5.2 spherical lens
5.3 spherical lens
51, 52 Additional light source
6 Substrate
7 Optical barrier
8 Aperture
9 Converter layer
12 Position of a wristwatch
12.1 Area of low arterial density
12.2 Area of medium arterial density
12.3 Area of high arterial density
13 Substrate
15 Bondpad
16 Emitter housing
17 Detector housing
18 VCSEL eye
19 Objective range
A Artery
V Vein
T Skin
Z Pulse measurement cycle
r red
g green
IR infrared
Y yellow
α Tilt angle

1. A vital sensor, in particular pulse sensor, comprising:
at least one pixelated emitter array with a first and at least one second pixel, which are each configured to emit light of a wavelength range in the direction of a projection surface;
at least one optical element which is arranged between the at least one pixelated emitter array and the projection surface and which is configured to direct light of the first pixel onto a first region of the projection surface and light of the at least one second pixel onto a second region of the projection surface which differs from the first region;
at least one photodetector configured to detect the light emitted by the pixels and reflected on the projection surface; and
an evaluation unit which is configured to control the first and the at least one second pixel in a pulsed and time-sequential manner in order to determine a first reference value.

2. The vital sensor according to claim 1, wherein the projection surface is formed by the skin of a human wearer of the vital sensor.

3. The vital sensor according to claim 2, wherein the evaluation unit is configured to control at least one of the first and the at least one second pixel for measuring a vital parameter, in particular the pulse rate, of the human wearer on the basis of the first reference value.

4. The vital sensor according to claim 3, wherein the evaluation unit is configured
to determine a second reference value during the measurement of the vital parameter, and to control the first and the at least one second pixel in a pulsed and time-sequential manner on the basis of the second reference value.

5. The vital sensor according to claim 1,
wherein the at least one optical element comprises at least one of the following:
a refractive lens, in particular a spherical lens;
a Fresnel step lens;
a diffractive optical element, in particular a diffractive lens; and
a lens of a metamaterial.

6. The vital sensor according to claim 1, wherein an aperture is arranged in front of each pixel of the pixelated emitter array, which is configured to limit the cross-section of the light emitted by the pixels in particular to a light spot.

7. The vital sensor according to claim 1, wherein the at least one optical element comprises at least one spherical lens.

8. The vital sensor according to claim 7, wherein at least two pixels of the pixelated emitter array are associated to the at least one spherical lens.

9. The vital sensor according to claim 1, wherein the at least one optical element comprises a number of spherical lenses corresponding to the number of pixels of the pixelated emitter array, wherein one spherical lens is associated with each pixel of the pixelated emitter array.

10. The vital sensor according to claim 7, wherein at least one spherical lens is arranged eccentrically with respect to at least one of the first- and the at least one second pixel as seen in an emission direction of the pixelated emitter array.

11. The vital sensor according to claim 1, wherein the first and the at least one second pixel are each formed by a Vertical Cavity Surface Emitting Laser (VCSEL).

12. The vital sensor according to claim 1, wherein a converter layer is arranged on at least one pixel of the pixelated emitter array, which is configured to at least partially convert the light emitted by the pixel into light of a different wavelength, in particular broadband light.

13. The vital sensor according to claim 1, wherein the first pixel is configured to emit light of a first wavelength and the second pixel is configured to emit light of a second wavelength different from the first wavelength.

14. The vital sensor according to claim 1, comprising a first and at least one second pixelated emitter array, wherein pixels of the first pixelated emitter array are configured to emit light of a first wavelength and pixels of the at least one second pixelated emitter array are configured to emit light of a second wavelength different from the first wavelength.

15. The vital sensor according to claim 1, comprising at least two photodetectors which are arranged symmetrically, in particular along a circular virtual line, around the at least one pixelated emitter array.

16. The vital sensor according to claim 2, wherein the evaluation unit is configured to apply a current derived from the first reference value to at least one of the first and the at least one second pixel for measuring a vital parameter, in particular the pulse rate, wherein in particular the current is greater than a current for determining the first reference value.

17. A method for measuring a vital parameter, in particular the pulse rate, of a human wearer of a vital sensor, in particular a pulse sensor, comprising the steps:
sequentially emitting pulsed light of a wavelength range by means of a first and at least one second pixel of a pixelated emitter array in the direction of the skin of the human wearer, wherein a light pulse generated by the first pixel is directed onto a first region of the skin of the human wearer and a light pulse generated by the second pixel is directed onto a second region of the skin of the human wearer which differs from the first region;
detecting the light emitted by the pixels and reflected from the skin of the human wearer by means of at least one photodetector; and
determining a first reference value for each pixel on the basis of which at least one of the first and the at least one second pixel is controlled to measure the vital parameter.

18. The method according to claim 17, further comprising determining a second reference value during the measurement of the vital parameter, wherein the first reference value is determined again for each pixel if the second reference value falls below a predefined threshold value.

19. The method according to claim 17, wherein for measuring the vital parameter only those pixels are controlled for which the first reference value exceeds a predefined threshold value.

20. The method according to claim 17, wherein the pixels for which the first reference value exceeds a predefined threshold value are provided with a higher current for measuring the vital parameter.

21. The method according to claim 17, wherein at least one optical element is arranged between the at least one pixelated emitter array and the skin of the human wearer, wherein the optical element is configured to direct the light pulse generated by the first pixel onto the first region of the skin of the human wearer and the light pulse generated by the second pixel onto the second region of the skin of the human wearer.
